SEQUENCE LISTING (20 sequences)

SEQ ID NO: 1 - DNA, 18 bp, Homo sapiens
actaccttcaactccatc

SEQ ID NO: 2 - DNA, 20 bp, Homo sapiens
tgatcttgatcttcattgtg

SEQ ID NO: 3 - DNA, 20 bp, Homo sapiens
acatcaaagctctgcagaaa

SEQ ID NO: 4 - DNA, 23 bp, Homo sapiens
ctgaataccttcccaaatagaac

SEQ ID NO: 5 - DNA, 16 bp, Homo sapiens
tgcgagcgctgcacat

SEQ ID NO: 6 - DNA, 24 bp, Homo sapiens
gcagcgtgtacttatccttcttca

SEQ ID NO: 7 - DNA, 22 bp, Homo sapiens
aatacctcagcctccagcagat

SEQ ID NO: 8 - DNA, 23 bp, Homo sapiens
tgcgtcacaccattgctattctt

SEQ ID NO: 9 - DNA, 20 bp, Homo sapiens
cgtggaacgtttttcctgtt

SEQ ID NO: 10 - DNA, 21 bp, Homo sapiens
tgtaggtgctgaaatcaaccc

SEQ ID NO: 11 - DNA, 23 bp, Mus musculus
ccctcacactcagatcatcttct

SEQ ID NO: 12 - DNA, 19 bp, Mus musculus
gctacgacgtgggctacag

SEQ ID NO: 13 - DNA, 21 bp, Mus musculus
ctccatgagctttgtacaagg

SEQ ID NO: 14 - DNA, 20 bp, Mus musculus
tgctgatgtaccagttgggg

SEQ ID NO: 15 - DNA, 21 bp, Mus musculus
cagctgggctgtacaaacctt

SEQ ID NO: 16 - DNA, 21 bp, Mus musculus
cattggaagtgaagcgtttcg

SEQ ID NO: 17 - DNA, 20 bp, Mus musculus
ccaagccttatcggaaatga

SEQ ID NO: 18 - DNA, 20 bp, Mus musculus
ttttcacaggggagaaatcg

SEQ ID NO: 19 - DNA, 20 bp, Mus musculus
ctttgcagctccttcgttgc

SEQ ID NO: 20 - DNA, 20 bp, Mus musculus
acgatggaggggaatacagc

(19) United States
(12) Patent Application Publication
(10) Pub. No.: US 2022/0265724 A1
(43) Pub. Date: Aug. 25, 2022

(54) CELL THERAPEUTIC AGENT FOR ANTI-INFLAMMATORY OR DAMAGED TISSUE REGENERATION COMPRISING PRUSSIAN BLUE NANOPARTICLES, AND METHOD FOR PREPARING THE SAME

(71) Applicant: Gwangju Institute of Science and Technology, Gwangju (KR)

(72) Inventors: Giyoong TAE, Gwangju (KR); Abhishek Sahu, Gwangju (KR); Hee Seok YANG, Cheonan (KR); Jin JEON, Cheonan (KR)

(21) Appl. No.: 17/571,116

(22) Filed: Jan. 7, 2022

(30) Foreign Application Priority Data
Jan. 8, 2021 (KR) 10-2021-0002528

(51) Int. Cl. A61K 35/28; A61K 9/14; A61K 33/26; A61P 39/06

(52) U.S. Cl. CPC A61K 35/28; A61K 9/14; A61K 33/26; A61P 39/06

(57) ABSTRACT

An embodiment of the present invention provides a cell therapeutic agent for anti-inflammatory or damaged tissue regeneration comprising Prussian blue nanoparticles and cells, and a method for preparing the same. According to an embodiment of the present invention, there is an effect capable of providing a cell therapeutic agent having improved therapeutic performance and improved oxidative stress resistance, engraftment rate and viability at a damage site through the introduction of Prussian blue nanoparticles having ROS scavenging ability and anti-inflammatory properties, and a method for preparing the same.

### BACKGROUND OF THE INVENTION

### Field of the Invention

The present invention relates to a cell therapeutic agent for anti-inflammatory or damaged tissue regeneration and a method for preparing the same, more particularly to a cell therapeutic agent for anti-inflammatory or damaged tissue regeneration comprising Prussian blue nanoparticles and a method for preparing the same.

### INCORPORATION BY REFERENCE

The sequence listing for this application has been submitted in accordance with 37 CFR § 1.821 in forms of ASCII text file containing the sequence listing file entitled “FUS-210048 Sequence Listing_revised draft.txt” created May 9, 2022, 3.26 kb. Applicants hereby incorporate by reference the sequence listing provided in forms of the ASCII text file into the present specification.

### Description of the Related Art

Ischemia/reperfusion (I/R) injury refers to a pathophysiological condition in which an organ or tissue undergoes blood flow recovery (reperfusion) after a period of hypoxia due to blood flow disturbance (ischemia). When blood supply to a tissue is cut off or lost and sudden ischemia occurs, the ischemic tissue malfunctions and necrosis occurs, and it is essential to supply oxygen and nutrients to these ischemic tissues and to resupply blood for regeneration. However, the tissue damage caused during reperfusion may be more severe than the damage caused during ischemia. Acute ischemia/reperfusion damage affects all organs and tissues of the human body and may lead to death in severe cases, and proper treatment thereof is thus important.

Recently, cell therapy via stem cell introduction has begun to emerge as a promising way to treat ischemia-reperfusion damage. The introduction of stem cells into the wound site may induce self-healing and regeneration of damaged cells or tissues. Practically, it has been studied that direct transplantation or systemic infusion of stem cells accelerates the endogenous recovery process in ischemia-reperfusion damage and decreases mortality in ischemia-reperfusion damage of the heart, liver, kidney, and intestine.

However, a damaged tissue site is a poor environment for stem cells to survive since inflammation or reactive oxygen species (ROS) are present in large amounts at the damaged tissue site. When stem cells are transplanted into these damaged tissue sites, there is a problem that the highly oxidative stress environment inhibits the survival and engraftment of the introduced stem cells and the treatment effect becomes significantly low, and research and development are required to improve this problem.

### CITATION LIST

### Patent Literature

Patent Literature 1: Korean Patent No. 1815187

### SUMMARY OF THE INVENTION

The technical object to be achieved by the present invention is to solve the problems of the prior art described above, and to provide a cell therapeutic agent having improved therapeutic performance and improved oxidative stress resistance, engraftment rate and viability at a damage site through the introduction of Prussian blue nanoparticles having ROS scavenging ability and anti-inflammatory properties.

The technical object to be achieved by the present invention is to provide a method for preparing a cell therapeutic agent for anti-inflammatory or damaged tissue regeneration, which can be simply prepared by only incubating cells and Prussian blue nanoparticles together without a special preparation method or cumbersome process.

The technical objects to be achieved by the present invention are not limited to the technical objects mentioned above, and other technical objects not mentioned will be clearly understood by those of ordinary skill in the art to which the present invention pertains from the following description.

In order to achieve the technical objects, an embodiment of the present invention provides a cell therapeutic agent for anti-inflammatory or damaged tissue regeneration.

The cell therapeutic agent for anti-inflammatory or damaged tissue regeneration may comprise Prussian blue nanoparticles; and cells.

In this case, the Prussian blue nanoparticles may exist by being impregnated into the inside of the cells.

The Prussian blue nanoparticles may have ROS scavenging properties, and the cells may have oxidative stress resistance improved by the Prussian blue nanoparticles.

The cells may be stem cells.

The stem cells may be mesenchymal stem cells.

The Prussian blue nanoparticles may have an average particle diameter of 10 nm to 200 nm.

For example, the Prussian blue nanoparticles may have an average particle diameter of 30 nm to 50 nm.

The cells may be incubated at a concentration of 25 μg/mL to 500 μg/mL of the Prussian blue nanoparticles.

The cells may be incubated together with the Prussian blue nanoparticles for 3 hours to 48 hours.

The therapeutic agent may be an injection formulation.

In order to achieve the technical objects, an embodiment of the present invention provides a method for preparing a cell therapeutic agent for anti-inflammatory or damaged tissue regeneration.

The method for preparing a cell therapeutic agent for anti-inflammatory or damaged tissue regeneration may comprise preparing Prussian Blue nanoparticles; incubating the Prussian blue nanoparticles and cells together so that the Prussian blue nanoparticles are impregnated into the inside of the cells; and obtaining cells containing Prussian blue nanoparticles from the step.

The cells may be incubated at a concentration of 25 μg/mL to 500 μg/mL of the Prussian blue nanoparticles in the step of incubating the Prussian blue nanoparticles and the cells together.

The cells may be incubated together with the Prussian Blue nanoparticles for 3 hours to 48 hours in the step of incubating the Prussian blue nanoparticles and the cells together.

### BRIEF DESCRIPTION OF THE DRAWINGS

FIG. 1 is fluorescence microscopy images acquired by staining MSC and PB-MSC cells prepared according to Preparation Examples and Comparative Examples with Calcein-AM and PI to confirm survival of the cells;

FIG. 2 is graphs illustrating the analyzed properties of MSC and PB-MSC cells prepared according to Comparative Examples and Preparation Examples of the present invention;

FIG. 3 is confocal fluorescence microscopy images for confirming the impregnation of PB into the inside of MSC and PB-MSC cells prepared according to Comparative Examples and Preparation Examples of the present invention;

FIG. 4 is graphs illustrating the pluripotency and multilineage differentiation of MSC and PB-MSC cells prepared according to Comparative Examples and Preparation Examples of the present invention;

FIG. 5 is graphs illustrating the in vitro paracrine activity and in vitro anti-inflammatory activity of MSC and PB-MSC cells prepared according to Comparative Examples and Preparation Examples of the present invention;

FIG. 6 is graphs and images illustrating the IRI therapeutic activity of MSC and PB-MSC cells prepared according to Comparative Examples and Preparation Examples of the present invention through serum analysis and histopathological characteristic analysis of injured liver tissue;

FIG. 7 is images illustrating the H&E-stained I/R-damaged liver tissue to confirm the IRI therapeutic activity of MSC and PB-MSC cells prepared according to Comparative Examples and Preparation Examples of the present invention;

FIG. 8 is images and graphs for confirming the in vivo antioxidant activity of MSC and PB-MSC cells prepared according to Comparative Examples and Preparation Examples of the present invention; and

FIG. 9 is graphs and images for confirming the in vivo anti-inflammatory activity of MSC and PB-MSC cells prepared according to Comparative Examples and Preparation Examples of the present invention.

### DETAILED DESCRIPTION OF THE PREFERRED EMBODIMENTS

Hereinafter, the present invention will be described with reference to the accompanying drawings. However, the present invention may be embodied in several different forms, and thus is not limited to the embodiments described herein. In order to clearly explain the present invention, parts irrelevant to the description are omitted in the drawings and similar reference numerals are attached to similar parts throughout the specification.

Throughout the specification, when a part is said to be “connected (linked, contacted, coupled)” with another part, this includes not only the case of being “directly connected” but also the case of being “indirectly connected” with another member interposed therebetween. In addition, when a part “includes” a certain component, this means that other components may be further provided but are not excluded unless otherwise stated.

The terms used herein are used only to describe specific embodiments, but are not intended to limit the present invention. The singular expression includes the plural expression unless the context clearly dictates otherwise. In the present specification, it should be understood that terms such as “comprise” or “have” are intended to designate that a feature, number, step, operation, component, part, or combination thereof described in the specification exists but do not preclude the possibility of addition or existence of one or more other features, numbers, steps, operations, components, parts, or combinations thereof.

Hereinafter, embodiments of the present invention will be described in detail with reference to the accompanying drawings.

A cell therapeutic agent for anti-inflammatory or damaged tissue regeneration according to an embodiment of the present invention will be described.

The cell may be stem cell.

The stem cell therapeutic agent for anti-inflammatory or damaged tissue regeneration may comprise Prussian blue nanoparticles; and stem cells.

As used herein, the term “stem cell therapeutic agent” refers to a therapeutic agent used for the tissue regeneration treatment, which is prepared by proliferating and selecting live autologous, allogenic, or xenogenic stem cells in vitro and introduced into the body in order to restore the tissue and function of cells.

A method in which a damaged tissue is treated by transplanting stem cells into the damaged tissue is a promising therapeutic method that enables self-healing and regeneration of damage sites through immunomodulation and paracrine effects of stem cells. However, when stem cells are transplanted into damaged tissue sites as described above, there is a problem that the highly oxidative stress environment of the damage sites decreases the survival and engraftment ability of the introduced stem cells and the treatment effect becomes low.

Based on this, in the present invention, a stem cell therapeutic agent having improved treatment efficiency through stem cells is developed by introducing Prussian blue nanoparticles into stem cells to improve the resistance of the stem cells to oxidative stress and thus improve the viability and engraftment ability in the poor environment of a damaged tissue site.

In this case, the Prussian blue nanoparticles may exist by being impregnated into the inside of the stem cells.

The Prussian blue nanoparticles may have ROS scavenging properties, and the stem cells may have oxidative stress resistance improved by the Prussian blue nanoparticles.

The Prussian blue nanoparticles (PB nanoparticles) are hydrates of iron ferrocyanide, have a blue color, have been conventionally used mainly for the treatment of patients exposed to cesium or thallium, or have been developed as a biocompatible contrast agent for magnetic resonance imaging (MRI), are also used for photothermal cancer treatment thanks to their function to convert near-infrared rays into heat, and have been approved by the FDA and the biostability thereof has already been proven.

The Prussian blue nanoparticles have anti-inflammatory activity through intracellular ROS scavenging ability and inhibition of inflammatory cytokine expression. For these properties, when the PB nanoparticles are introduced into stem cells as in the present invention, not only the resistance of stem cells to oxidative stress is improved by the ROS scavenging ability and anti-inflammatory activity of the PB nanoparticles but also this increases the engraftment ability and viability of stem cells in an oxidative stress environment due to inflammation or reactive oxygen species (ROS) present in large amounts at the damaged tissue site and the effect of cell therapy can be thus suitably improved.

In particular, the stem cell therapeutic agent for anti-inflammatory or damaged tissue regeneration of the present invention can improve the treatment efficiency of ischemia-reperfusion injury by protecting stem cells from inflammation or reactive oxygen species present in large amounts at the damage site and assisting the engraftment and survival of stem cells in order to treat ischemia-reperfusion injury.

In this case, the stem cells may be mesenchymal stem cells.

In general, stem cells may be classified into adult stem cells, embryonic stem cells, dedifferentiated stem cells, and the like. Among these, adult stem cells are cells that exist in various organs of our body and play a regenerative action when the body is injured, and representatively include hematopoietic stem cells, mesenchymal stem cells, and the like, which are found in bone marrow, umbilical cord, and the like.

Among these, mesenchymal stem cells (MSC), which have advantages such as safety, standardization of separation and incubation technology, and low cost in mass production compared to other stem cells, may be most easily used for stem cell regeneration treatment and are thus most preferred in the present invention, but the stem cells are not limited thereto, and any known stem cells that can be used for the treatment of tissue damage may be used without limitation.

The Prussian blue nanoparticles may have an average particle diameter of 30 nm to 50 nm, most preferably an average particle diameter of 40 nm.

The stem cells may be incubated at a concentration of 25 μg/mL to 500 μg/mL, more preferably 100 μg/mL to 300 μg/mL, most preferably 200 μg/mL of the Prussian blue nanoparticles.

It is not preferable that the concentration of the Prussian blue nanoparticles is less than 25 μg/mL since the effect of protecting stem cells and the effect of improving the resistance of stem cells to oxidative stress by the Prussian blue nanoparticles are insignificant.

It is not preferable that the concentration of the Prussian blue nanoparticles is 500 μg/mL or more since the viability of stem cells may decrease.

Consequently, the stem cells of the present invention may be incubated at a concentration of 25 μg/mL to 500 μg/mL of the Prussian blue nanoparticles, and are incubated at a concentration of more preferably 100 μg/mL to 300 μg/mL, most preferably 200 μg/mL of the Prussian blue nanoparticles.

The stem cells may be incubated together with the Prussian blue nanoparticles for 3 hours to 48 hours, most preferably for 24 hours.

It is not preferable that the incubation time is less than 3 hours since the Prussian blue nanoparticles are not sufficiently impregnated into the inside of stem cells and thus the effect of protecting stem cells and the effect of improving the resistance of stem cells to oxidative stress are insignificant.

It is not efficient that the incubation time is more than 48 hours since the Prussian blue nanoparticles impregnated into the inside of stem cells do not increase more than a certain level even if the incubation time is increased more than this.

Consequently, the stem cells are incubated together with the Prussian blue nanoparticles preferably for 3 hours to 48 hours, most preferably for 24 hours.

The stem cell therapeutic agent may be an injection formulation, but is not limited thereto, and any formulation may be used without limitation as long as it is a proper formulation and has properties for use as a stem cell therapeutic agent.

For the characteristics of the configuration as described above, according to an embodiment of the present invention, there is an effect capable of providing a stem cell therapeutic agent having improved oxidative stress resistance, engraftment rate and viability at a damage site through the introduction of Prussian blue nanoparticles having ROS scavenging ability and anti-inflammatory properties.

A method for preparing a cell therapeutic agent for anti-inflammatory or damaged tissue regeneration according to another embodiment of the present invention will be described.

The cell may be stem cell.

In this case, some of the components of the embodiment are the same as the components of the above-described embodiment, and thus the description of the same components will be omitted or simply described, and the added components will be mainly described.

The method for preparing a stem cell therapeutic agent for anti-inflammatory or damaged tissue regeneration may comprise preparing Prussian Blue nanoparticles; incubating the Prussian blue nanoparticles and stem cells together so that the Prussian blue nanoparticles are impregnated into the inside of the stem cells; and obtaining stem cells containing Prussian blue nanoparticles from the step.

The stem cells may be incubated at a concentration of 25 μg/mL to 500 μg/mL of the Prussian blue nanoparticles in the step of incubating the Prussian blue nanoparticles and the stem cells together.

It is not preferable that the concentration of the Prussian blue nanoparticles is less than 25 μg/mL since the effect of protecting stem cells and the effect of improving the resistance of stem cells to oxidative stress by the Prussian blue nanoparticles are insignificant.

It is not preferable that the concentration of the Prussian blue nanoparticles is 500 μg/mL or more since the viability of stem cells may decrease.

Consequently, in the step of incubating the Prussian blue nanoparticles and the stem cells together, the stem cells are incubated at a concentration of preferably 25 μg/mL to 500 μg/mL, more preferably 100 μg/mL to 300 μg/mL, most preferably 200 μg/mL of the Prussian blue nanoparticles.

The stem cells may be incubated together with the Prussian Blue nanoparticles for 3 hours to 48 hours in the step of incubating the Prussian blue nanoparticles and the stem cells together.

It is not preferable that the incubation time is less than 3 hours since the Prussian blue nanoparticles are not sufficiently impregnated into the inside of the stem cells and thus the effect of protecting stem cells and the effect of improving the resistance of stem cells to oxidative stress are insignificant.

It is not efficient that the incubation time is more than 48 hours since the Prussian blue nanoparticles impregnated into the inside of stem cells do not increase more than a certain level even if the incubation time is increased more than this.

Consequently, in the step of incubating the Prussian blue nanoparticles and the stem cells together, the stem cells are incubated together with the Prussian blue nanoparticles preferably for 3 hours to 48 hours, most preferably for 24 hours.

For the characteristics of the configuration as described above, according to an embodiment of the present invention, there is an effect capable of providing a method for preparing a stem cell therapeutic agent for anti-inflammatory or damaged tissue regeneration, which can be simply prepared by only incubating stem cells and Prussian blue nanoparticles together without a special preparation method or cumbersome process.

Hereinafter, the present invention will be described in more detail with reference to Preparation Examples, Comparative Examples and Experimental Examples. However, the present invention is not limited to the following Preparation Examples and Experimental Examples.

<Preparation Examples 1 to 5> Preparation of mesenchymal stem cells containing Prussian blue nanoparticles (PB-MSC)

In order to prepare a possible stem cell therapeutic agent for anti-inflammatory or damaged tissue regeneration according to an embodiment of the present invention, mesenchymal stem cells impregnated with Prussian blue nanoparticles were prepared.

In order to prepare the mesenchymal stem cells impregnated with Prussian blue nanoparticles, PB nanoparticles having an average size of up to 40 nm were first synthesized. Next, human bone marrow-derived mesenchymal stem cells (MSCs) were prepared by incubating the cells in MEMα supplemented with 10% FBS and 1% antibiotics and antifungals at 37° C. in a 5% CO₂ atmosphere. Next, the MSCs were seeded in 96-well cell culture plates and incubated for 24 hours together with PB nanoparticles at 25 μg/mL, 50 μg/mL, 100 μg/mL, 200 μg/mL, or 500 μg/mL.

### <Preparation Examples 6 to 10> Preparation of Mesenchymal Stem Cells Containing Prussian Blue Nanoparticles (PB-MSC)

Preparation Examples 6 to 10 were prepared under the same process conditions as in Preparation Example 1 except that the incubation time in Preparation Example 1 was set to 48 hours.

### <Comparative Example 1> Preparation of Control Mesenchymal Stem Cells

Comparative Example 1 was prepared under the same process conditions as in Preparation Example 1 except that PB nanoparticles in Preparation Example 1 were not added (0 μg/mL) during the incubation of MSCs.

### <Comparative Example 2> Preparation of Control Mesenchymal Stem Cells

Comparative Example 2 was prepared under the same process conditions as in Preparation Example 6 except that PB nanoparticles in Preparation Example 6 were not added (0 μg/mL) during the incubation of MSCs.

### <Experimental Example 1> Experiment to Confirm Biocompatibility of PB Nanoparticles

An experiment was conducted to confirm the biocompatibility of the PB nanoparticles of the present invention. To this end, the stem cells incubated according to Preparation Examples and Comparative Examples were first washed and incubated in a new medium containing MTT reagent at 37° C. for 2 hours. Finally, the formazan crystals formed inside the cells were dissolved in DMSO, and the absorbance was measured at 590 nm using the Varioskan™ LUX microplate reader (Thermo-Fischer Scientific, Waltham, Mass., USA). At this time, the survival of the cells was confirmed by incubating the cells together with a dye containing Calcein-AM and PI for 30 minutes. After staining, live (green) cells and dead (red) cells were observed under a fluorescence microscope (Nikon TE2000-U, Tokyo, Japan).

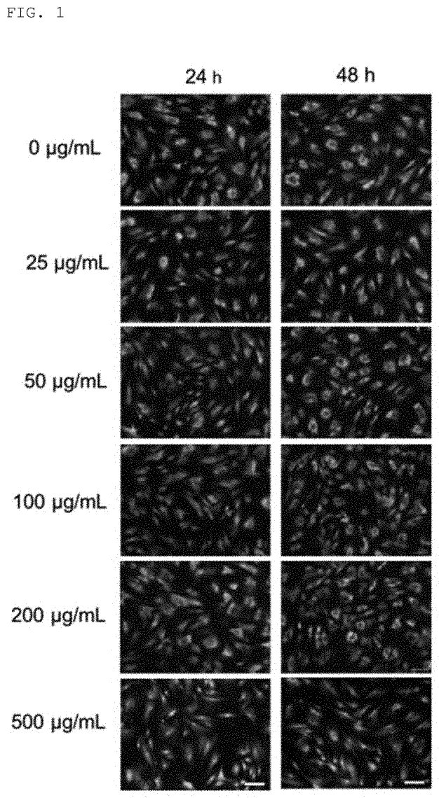

FIG. 1 is fluorescence microscopy images acquired by staining MSC cells treated with PB nanoparticles of the present invention, which are prepared according to Preparation Examples and Comparative Examples, with Calcein-AM and PI to confirm survival of the cells.

Referring to FIG. 1, it can be seen that almost all MSC cells treated with PB nanoparticles are viable through the bright green fluorescence signal corresponding to Calcein-AM staining, and the biocompatibility of the PB nanoparticles has been confirmed through this. However, a red fluorescence signal corresponding to dead cells is observed when MSC cells are treated with PB nanoparticles at 500 μg/mL or more, and thus it has also been confirmed that PB nanoparticles at 500 μg/mL or more exhibit an adverse effect on MSC cell survival.

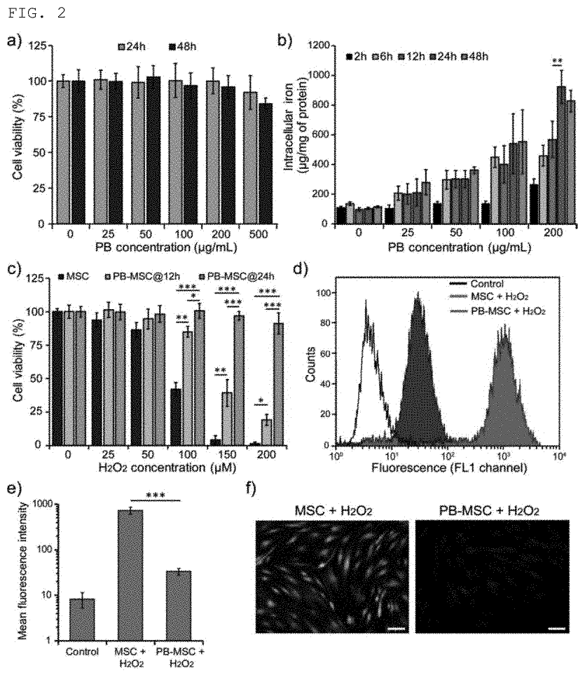

FIG. 2 is graphs illustrating the analyzed properties of PB-MSC cells prepared according to Comparative Examples and Preparation Examples of the present invention.

(a) of FIG. 2 is a graph illustrating the viability of the PB-MSC cells prepared according to Comparative Examples and Preparation Examples.

Referring to (a) of FIG. 2, it can be seen that a significant decrease is not observed in the viability of MSCs after being incubated together with PB for 24 hours or 48 hours. It can be seen that MSCs are nearly 100% viable and metabolically active in the presence of PB nanoparticles at up to 200 μg/mL and the cell viability of MSCs slightly decreases to 84±4% after being incubated with PB (500 μg/mL) at a greatly high concentration for 48 hours. Consequently, it has been confirmed that the treatment with PB nanoparticles at 500 μg/mL or less does not affect the viability of MSCs.

(b) of FIG. 2 is a graph for confirming the impregnation of PB nanoparticles into PB-MSC cells depending on the time of treatment with PB nanoparticles through the analysis of iron ion in the PB nanoparticles.

Referring to (b) of FIG. 2, it can be seen that the intracellular iron concentration increases not only as the incubation time increases but also as the PB concentration increases. It can be seen that MSCs incubated together with PB at 200 μg/mL for 24 hours exhibited the highest intracellular iron content, that is, the iron level is about 5 times the normal iron level in MSCs. On the other hand, the intracellular iron level does not increase any more when the incubation time is increased from 24 hours to 48 hours, so it can be seen that incubation of MSCs together with PB at 200 μg/mL for 24 hours is the optimal condition for impregnating PB nanoparticles into MSCs.

### <Experimental Example 2> Experiment to Confirm Impregnation of Mesenchymal Stem Cells with Prussian Blue Nanoparticles

An experiment was conducted to confirm the impregnation of MSCs with the PB nanoparticles of the present invention. To this end, cells according to Preparation Examples and Comparative Examples incubated at 37° C. were first prepared, and the cells were washed and lysed with 1 N sodium hydroxide solution. The lysed sample was analyzed by quantification of intracellular iron concentration by ferrozine-based colorimetric assay.

The intracellular uptake of PB nanoparticles was analyzed by confocal fluorescence microscopy. Propidium iodide (PI), a positively charged fluorescent dye, was encapsulated into negatively charged PBs through electrostatic interaction during nanoparticle synthesis. PI-encapsulated PB nanoparticles were incubated together with MSCs for 2 hours and washed for imaging.

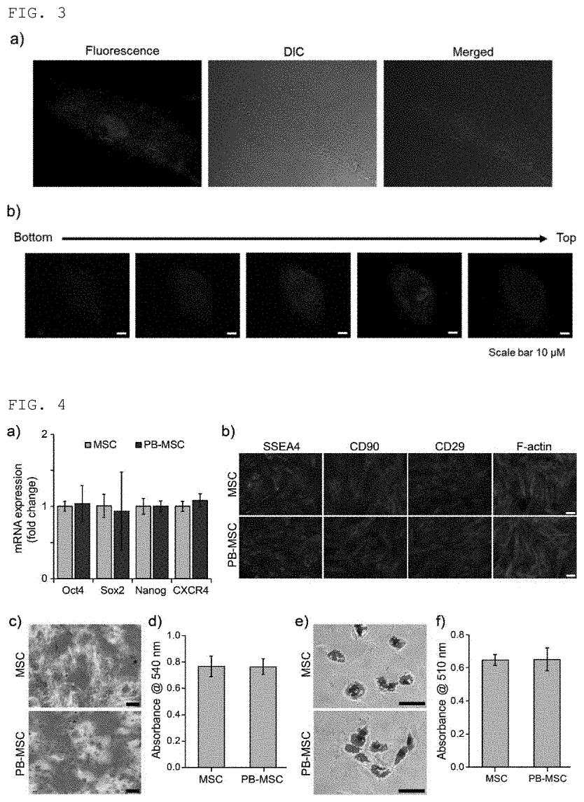

FIG. 3 is confocal fluorescence microscopy images for confirming the impregnation of PB into the inside of PB-MSC cells prepared according to Comparative Examples and Preparation Examples of the present invention.

Referring to (a) of FIG. 3, it can be seen that the intracellular PI fluorescence signal is clearly observed throughout the cytoplasm of the cells ((a) of FIG. 3). Since free PI is impermeable to living cells, the intracellular fluorescence signal is attributed to the PI-encapsulated PB nanoparticles, and thus the intracellular uptake of PB nanoparticles may be confirmed through this.

Referring to (b) of FIG. 3, it can be seen that the fluorescence signal is mostly in the central area of the cell. Through this, it can be seen that the PB nanoparticles are not bound only to the cell surface but are mostly impregnated into the inside of the cells.

### <Experimental Example 3> Experiment to Measure Oxidative Stress Resistance and Intracellular ROS Scavenging Ability

An experiment was conducted to confirm the oxidative stress resistance and intracellular ROS scavenging ability of MSC and PB-MSC cells according to Comparative Examples and Preparation Examples of the present invention. Hydrogen peroxide (H₂O₂) is a classic ROS that is elevated in a large number of inflammation-associated oxidative stress conditions including liver I/R injury. Therefore, the effect of H₂O₂ at various concentrations on PB-MSCs was investigated and compared with that on MSCs not impregnated with PB nanoparticles.

First, oxidative stress was induced by exposing cells to hydrogen peroxide (H₂O₂) at various concentrations. After being incubated for 24 hours, the cells were washed and the cell metabolic activity was measured through MTT assay to quantify the viability.

The intracellular ROS was measured using DCFH-DA, a ROS-sensitive fluorescent probe. MSCs and PB-MSCs were grown in 12-well cell culture plates and exposed to 100 μM H₂O₂ for 2 hours. Thereafter, the cells were washed and incubated together with 10 μM DCFH-DA in serum-free medium at 37° C. for 30 minutes. After being incubated, the cells were washed with PBS, trypsinized, and centrifuged at 1500 rpm for 3 minutes at 4° C. to collect the cells. The collected cells were analyzed using a flow cytometer (FACSCalibur, BD Biosciences, San Jose, Calif., USA). MSCs not treated with H₂O₂ were used as a control. The intracellular fluorescence signal was visualized and imaged with blue excitation light (488 nm) using a fluorescence microscope (TE2000, Nikon, Tokyo, Japan).

Two different PB-MSCs of Preparation Example 4 (PB-MSC@12 h) and Preparation Example 9 (PB-MSC@24 h) were used in the experiment to analyze the effect of PB nanoparticles impregnation on H₂O₂-mediated oxidative stress.

Referring to (c) of FIG. 2, it can be seen that the metabolic activity of the MSCs not impregnated with PB nanoparticles of Comparative Example decreases as the H₂O₂ concentration increases. In contrast, it can be seen that the PB-MSC cells of Preparation Example exhibit more favorable cell viability in response to the treatment with H₂O₂. At low H₂O₂ concentrations (25 and 50 μM), all three groups exhibited high (>90%) metabolic activity, and there was no significant difference in metabolic activity between groups. However, at 100 μM H₂O₂ concentration, the viability of MSCs of Comparative Example greatly decreased to approximately 40% but the viability of the PB-MSC cells of Preparation Examples was confirmed to be 85% and 100%, respectively. It can be seen that the MSCs of Comparative Example lose the cell viability by 90% or more when H₂O₂ was increased to 150 μM and 200 μM, but PB-MSC@24 h exhibits a cell viability of 90% or more even in the presence of 200 μM H₂O₂. Consequently, it has been confirmed that the MSCs not impregnated with PB nanoparticles of Comparative Example are significantly vulnerable to an environment having a high ROS level but the PB-MSCs impregnated with PB nanoparticles of the present invention exhibit improved viability in an environment having a high ROS level as the PB-MSCs are impregnated with a larger amount of PB nanoparticles.

In the subsequent experiment, the experiment was conducted using the cells of Preparation Example 9 (PB-MSC@24 h). Hereinafter, the cells of Preparation Example 9 will be thus simply referred to as PB-MSCs.

The intracellular PB nanoparticles of PB-MSCs can reduce H₂O₂-mediated high oxidative stress through the excellent ROS scavenging properties. In order to confirm this, the intracellular ROS level was quantified by flow cytometry using DCFH-DA as a fluorescent probe.

Referring to (d) to (f) of FIG. 2, it can be seen that the MSCs treated with 100 μM H₂O₂ for 4 hours of Comparative Example exhibit a high ROS level and a considerably high intracellular DCFH fluorescence signal because of the highly oxidative stress environment inside the cells but the PB-MSCs of Example exhibit remarkably decreased ROS level and intracellular DCFH fluorescence signal, and this is also consistent with the results of fluorescence imaging by flow cytometry. Consequently, it has been confirmed that intracellular PB nanoparticles exhibit an effect capable of protecting MSC cells even in an environment containing an excessive amount of H₂O₂ through ROS scavenging and oxidative stress reduction.

### <Experimental Example 4> Experiment to Confirm Pluripotency and Multilineage Differentiation

An experiment was conducted to confirm the pluripotency and multilineage differentiation of the PB-MSC cells of the present invention. Pluripotency and multilineage differentiation are key properties of MSC therapeutic agents. Therefore, the pluripotency and multilineage differentiation of MSCs should not be affected by the impregnation with PB nanoparticles. To this end, the expression of pluripotent marker genes (Oct4, Sox2, Nanog and CXCR4) was first analyzed using RT-qPCR. The expression of all the genes was normalized to the expression of β-actin. The expression of pluripotent genes in PB-MSCs of Preparation Example was compared with that in MSCs of Comparative Example by AACt method. The primer sequences of β-actin, Oct4, Sox2, Nanog and CXCR4 are presented in Table 1 below.

TABLE 1

| Gene | Primers |
|---|---|
| β-actin | Forward: 5′-ACTACCTTCAACTCCATC-3′ |
| (human) | Reverse: 5′-TGATCTTGATCTTCATTGTG-3′ |
| Oct4 | Forward: 5′-ACATCAAAGCTCTGCAGAAA-3′ |
| (human) | Reverse: 5′-CTGAATACCTTCCCAAATAGAAC-3′ |
| Sox2 | Forward: 5′-TGCGAGCGCTGCACAT-3′ |
| (human) | Reverse: 5′-GCAGCGTGTACTTATCCTTCTTCA-3′ |
| Nanog | Forward: 5′-AATACCTCAGCCTCCAGCAGAT-3′ |
| (human) | Reverse: 5′-TGCGTCACACCATTGCTATTCTT-3′ |
| CXCR4 | Forward: 5′-CGTGGAACGTTTTTCCTGTT-3′ |
| (human) | Reverse: 5′-TGTAGGTGCTGAAATCAACCC-3′ |
| TNF-α | Forward: 5′-CCCTCACACTCAGATCATCTTCT-3′ |
| (mouse) | Reverse: 5′-GCTACGACGTGGGCTACAG-3′ |
| IL-1β | Forward: 5′-CTCCATGAGCTTTGTACAAGG-3′ |
| (mouse) | Reverse: 5′-TGCTGATGTACCAGTTGGGG-3′ |
| iNOS | Forward: 5′-CAGCTGGGCTGTACAAACCTT-3′ |
| (mouse) | Reverse: 5′-CATTGGAAGTGAAGCGTTTCG-3′ |
| IL-10 | Forward: 5′-CCAAGCCTTATCGGAAATGA-3′ |
| (mouse) | Reverse: 5′-TTTTCACAGGGGAGAAATCG-3′ |
| β-actin | Forward: 5′-CTTTGCAGCTCCTTCGTTGC-3′ |
| (mouse) | Reverse: 5′-ACGATGGAGGGGAATACAGC-3′ |

Next, cell pluripotent markers (SSEA4, CD90, CD29, F-actin) were specified for MSCs of Comparative Example and PB-MSCs of Preparation Example through immunofluorescence staining. To this end, the incubated cells were fixed in 10% neutral buffered formalin for 10 minutes and maintained with 1% BSA at room temperature for 60 minutes, and then the cells were rinsed two times with wash buffer (PBS+0.05%) and incubated overnight together with each primary antibody (SSEA4 (1:50) (Santa Cruz Biotechnology, USA), CD90 (1:100) (BD Bioscience, USA), or CD29 (1:100) (Abcam, UK)). The cells were then washed, and each secondary antibody (Alexa fluor 488-anti mouse IgG for SSEA4 and CD90 or Alexa fluor 594-anti rabbit IgG for CD29) was added thereto to be 1:200. After being incubated at room temperature for 2 hours, the cells were washed and counterstained with DAPI. For F-actin staining, the cells were incubated at 4° C. overnight with Alexa fluor 594 conjugated Phalloidin (1:50). After the cells were washed, the cell nuclei were also counterstained with DAPI. The cells were visualized and imaged using a fluorescence microscope (TE2000, Nikon, Tokyo, Japan).

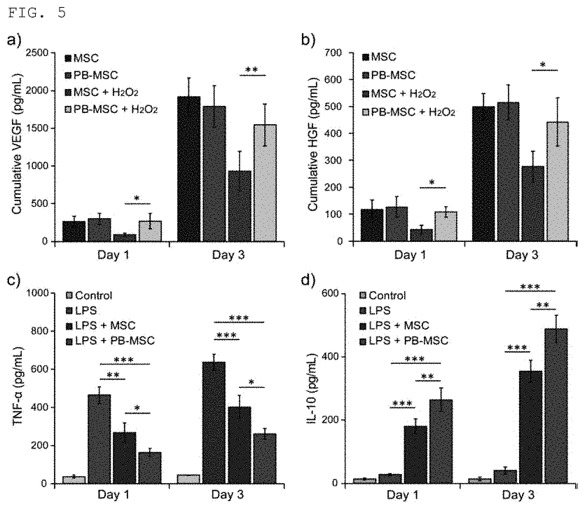

FIG. 4 is graphs illustrating the pluripotency and multilineage differentiation of PB-MSC cells prepared according to Comparative Example and Preparation Example of the present invention.

(a) of FIG. 4 is a graph illustrating the expression of pluripotent marker genes in the MSCs of Comparative Example and the PB-MSCs of Preparation Example for comparison.

Referring to (a) of FIG. 4, it can be seen that the expression of all the four genes in the PB-MSCs of Preparation Example of the present invention is similar to the expression of all the four genes in the normal MSCs of Comparative Example.

(b) of FIG. 4 is immunostaining images of SSEA4, CD90, CD29 and F-actin in the MSCs of Comparative Example and the PB-MSCs of Preparation Example.

Referring to (b) of FIG. 4, it can be seen that there is also no difference in the immunofluorescence staining between the MSCs of Comparative Example and the PB-MSCs of Preparation Example. Likewise, it can be seen that there is also no difference in the case of F-actin.

Consequently, through the gene expression and immunofluorescence imaging results of (a) and (b) of FIG. 4, it has been confirmed that the impregnation with PB nanoparticles does not adversely affect the cytoskeleton and pluripotency of MSCs.

For comparison of multilineage differentiation between the MSCs of Comparative Example and the PB-MSCs of Preparation Example, the cells were first inoculated into 24-well tissue culture plates (2×10⁴ cells/well). After the cell growth reached 80% to 90%, the medium was replaced with an osteogenic or adipogenic differentiation medium.

First, the basal medium was supplemented with dexamethasone (10 nM), β-glycerophosphate (10 mM) and L-ascorbic acid (50 μg/mL) for osteogenesis. After being incubated for 17 days, the cells were stained with Alizarin Red S (ARS) to assess calcium deposit formation. For quantification of staining, 10% (w/v) cetylpyridinium chloride solution was added to each well and the cells were incubated at room temperature for 30 minutes while shaking the plate. The absorbance of the dissolved dye was measured at 540 nm.

Next, for adipogenesis, the basal medium was replaced with StemPro adipogenic differentiation induction medium (Thermo Fischer Scientific, Waltham, Mass., USA). After induction for 21 days, the lipid droplets of differentiated cells were stained with Oil Red 0 (ORO). The stained cells were observed under a bright field microscope. For quantitative analysis, the stained ORO dye was extracted using 1000 μL of isopropanol and the absorbance was measured at 510 nm.

(c) of FIG. 4 is an image illustrating the MSCs and PB-MSCs stained with Alizarin Red S (ARS) after osteogenic differentiation.

(d) of FIG. 4 is an absorbance graph for quantitative analysis of the extracted ARS dye.

(e) of FIG. 4 is an image illustrating the MSCs and PB-MSCs stained with Oil Red 0 (ORO) after adipogenic differentiation.

(f) of FIG. 4 is an absorbance graph for quantitative analysis of the extracted ORO dye.

Referring to (c) to (f) of FIG. 4, it can be seen that PB-MSCs also successfully form a bone and produces lipid droplets at levels similar to those by MSCs. Through this, it has been confirmed that the impregnation of MSCs with PB nanoparticle of the present invention does not adversely affect the multilineage differentiation of cells.

### <Experimental Example 5> Experiment to Measure In Vitro Paracrine Activity

An experiment was conducted to measure the paracrine activity of the PB-MSC cells of the present invention. Stem cell-based cell therapy is due to the paracrine activity of the cells. When injected into damaged tissue, MSCs secrete various growth factors, cytokines and chemokines in response to environmental signals to promote tissue repair. In the present Experimental Example 5, an experiment was conducted to measure the paracrine activity of PB-MSC cells in order to analyze the effect of impregnation with PB nanoparticles on the paracrine activity of MSCs.

To this end, MSC or PB-MSC cells were first seeded in 24-well tissue culture plates and incubated for 24 hours. After that, the cells were washed with PBS, fresh medium was added thereto, the cells were incubated for 24 or 72 hours, and then the medium was collected, centrifuged (3000 rpm, 5 minutes, 4° C.) to remove cell debris, and stored at −20° C.

In order to confirm the effect of high oxidative stress on the activity, MSCs and PB-MSCs were exposed to 200 μM H₂O₂ and incubated for 2 hours, and then the cells were washed with PBS and incubated in a normal medium. The concentrations of VEGF and HGF secreted by the cells exposed to H₂O₂ were measured by ELISA.

FIG. 5 is graphs illustrating the in vitro paracrine activity and in vitro anti-inflammatory activity of PB-MSC cells prepared according to Comparative Example and Preparation Example of the present invention.

Referring to (a) and (b) of FIG. 5, it can be seen that MSCs and PB-MSCs both secrete similar levels of VEGF and HGF in a normal environment but secrete greatly decreased levels of VEGF and HGF after being exposed to an environment having a high H₂O₂ level. In contrast, it can be seen that the H₂O₂-treated PB-MSCs secrete significantly higher levels of VEGF and HGF compared to the MSCs of Comparative Example exposed to H₂O₂. Consequently, it has been confirmed that the impregnation with PB nanoparticles does not adversely affect the paracrine activity of MSCs.

### <Experimental Example 6> Experiment to Measure In Vitro Anti-Inflammatory Activity

An experiment was conducted to measure the in vitro anti-inflammatory activity of the PB-MSC cells of the present invention. LPS activates macrophages through toll-like receptor 4 (TLR4) and triggers a series of signaling events to produce ROS and other pro-inflammatory mediators such as TNF-α. Therefore, the anti-inflammatory activity of the PB-MSCs of the present invention was measured by measuring TNF-α secreted by RAW264.7 macrophages stimulated by LPS.

To this end, RAW 254.7 murine macrophages were first inoculated into 12-well plates at a concentration of 2×10⁴ cells per well. After 12 hours, macrophages were classified into the following groups depending on the treatment method: i) macrophages incubated in a normal medium; ii) macrophages activated with 100 ng/mL LPS for 12 hours and then incubated in a normal medium; and iii) macrophages activated with 100 ng/mL LPS for 12 hours and then co-incubated with MSCs or PB-MSCs. For analysis, the culture medium was collected after 24 or 72 hours of co-incubation. The concentrations of TNF-α and IL-10 secreted by macrophages were measured by ELISA (Thermo Fischer Scientific, Waltham, Mass., USA).

Referring to (c) and (d) of FIG. 5, it can be seen that the unstimulated control macrophages corresponding to group i) produce a significantly low level of TNF-α but the activated macrophages corresponding to group ii) produce a significantly high level of TNF-α. In group iii) incubated together with MSCs, it can be seen that the activated macrophages incubated together with MSCs according to Comparative Example secrete a slightly decreased level of TNF-α and an increased level of IL-10, an anti-inflammatory cytokine. The activated macrophages co-incubated with PB-MSCs secrete a significantly low level of TNF-α and a high level of IL-10. Through this, it has been confirmed that the anti-inflammatory function of stem cells is improved when MSCs are impregnated with PB nanoparticles.

### <Experimental Example 7> Experiment to Confirm In Vivo Therapeutic Activity

An experiment was conducted to confirm the in vivo therapeutic activity, particularly the ischemia-reperfusion injury (IRI) therapeutic activity in vivo of the PB-MSC cells of the present invention.

To this end, partially IRI-induced mice were first prepared. The IRI induction was conducted as follows.

First, mice were anesthetized by intraperitoneal injection of a mixed solution of Ketamine and Xylazine (4:1 ratio). Next, the hepatic artery and the first branch of the portal vein were fixed with microvascular clamps except for the vessels in the right lower lobe to induce ischemic injury to about 70% of the liver. After ischemia for 90 minutes, microvascular clamps were removed to initiate reperfusion. At this time, the mice were prepared by dividing into four groups: PBS (500 μL) single injection, PB nanoparticles (50 μg/mouse) single injection, MSC (1×10⁵ cells/mouse) injection, and PB-MSC (1×10⁵ cells/mouse) injection. Mice in the Sham group underwent the same surgery but did not progress to vascular occlusion. After reperfusion for 3, 6, and 12 hours, respectively, a small amount of blood was collected, serum was separated for biochemical analysis, and whole blood and liver tissue were obtained for further analysis.

### <Experimental Example 7-1> Experiment to Analyze Serum of I/R Injured Liver Tissue

In order to measure the IRI therapeutic activity of the PB-MSC cells of the present invention, an experiment was first conducted to analyze the serum. To this end, alanine aminotransferase (ALT) and aspartate aminotransferase (AST) enzymes, which were common markers for confirming liver damage, were first measured.

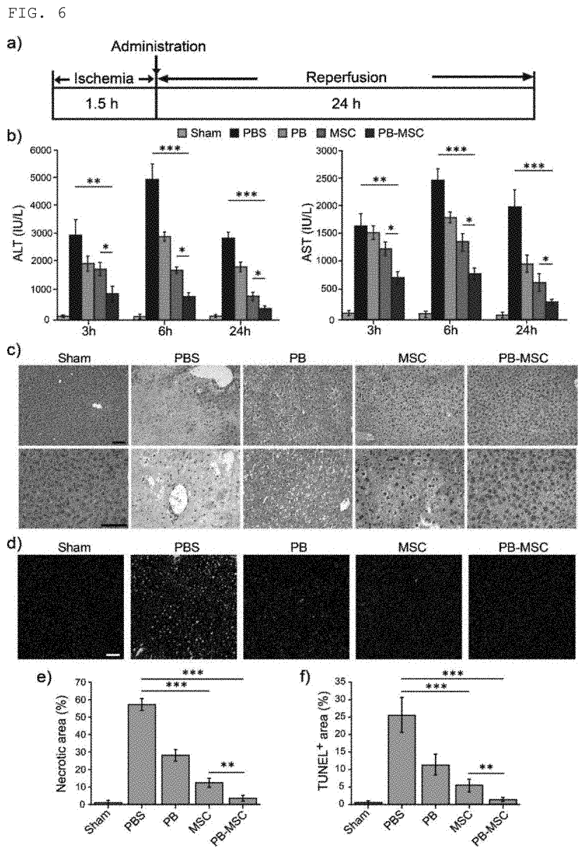

FIG. 6 is graphs and images illustrating the IRI therapeutic activity of the PB-MSC cells of the present invention through serum analysis and histopathological characteristic analysis of damaged liver tissue.

A mouse IRI model was constructed as illustrated in (a) of FIG. 6.

(b) of FIG. 6 is graphs illustrating the ALT and AST levels in serum, which are indicators of liver damage due to ischemia-reperfusion in a mouse IRI model.

Referring to (b) of FIG. 6B, the mice in the Sham group exhibit minimal serum ALT and AST levels at all time points, and the serum concentrations of ALT and AST in the PBS group are significantly high. In other words, it can be seen that this is a signal of liver damage due to ischemia-reperfusion. Compared with the PBS group, the mice treated with PB nanoparticles alone exhibit decreased serum levels of ALT and AST, indicating PB nanoparticles themselves also exhibit some therapeutic activity due to their ROS scavenging ability. However, the mice treated with PB-MSCs exhibit the lowest levels of serum ALT/AST at all time points. Through this, it has been confirmed that PB-MSCs exhibit the most favorable effect of protecting the liver from liver tissue cell damage caused by ischemia-reperfusion injury.

### <Experimental Example 7-2> Experiment to Analyze Histopathological Characteristic of I/R Injured Liver Tissue

In order to measure the IRI therapeutic activity of the PB-MSC cells of the present invention, an experiment was conducted to analyze the histological and immunohistochemical characteristics of liver tissue damaged by IRI.

To this end, the collected liver tissue was fixed in 4% paraformaldehyde, embedded in paraffin, sectioned to a thickness of 6 μm, and stained with hematoxylin and eosin (H&E). Liver tissue damage was estimated by quantifying the necrotic areas in H&E-stained sections. Apoptosis in liver tissue was analyzed using a terminal deoxynucleotidyl transferase dUTP nick-end labeling (TUNEL) staining kit. TUNEL-positive cells were quantified using Image J software (version 1.8.0) in 4 to 6 randomly selected sections per liver.

(c) of FIG. 6 is images illustrating the H&E-stained liver tissue after I/R injury and various treatments.

(d) of FIG. 6 is images illustrating TUNEL-stained liver sections.

(e) of FIG. 6 is a graph illustrating the necrotic areas in the liver sections quantified from the H&E-stained images.

(f) of FIG. 6 is a graph illustrating the apoptosis in the liver tissue quantified from the TUNEL-stained images.

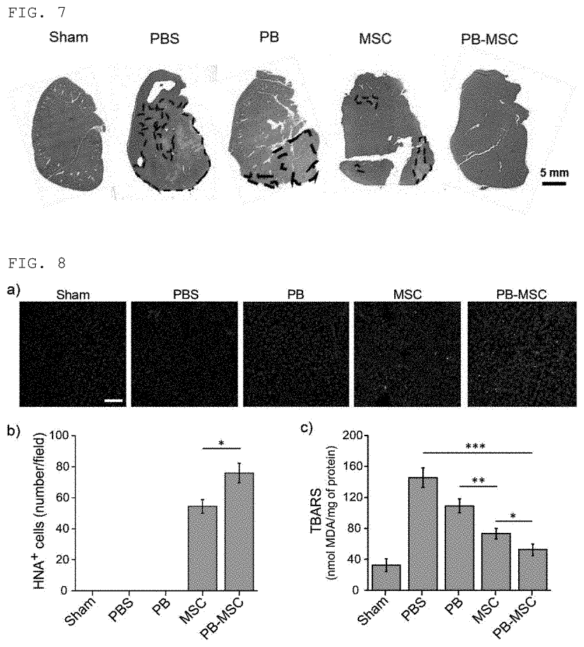

FIG. 7 is images illustrating the H&E-stained I/R-injured liver tissue to confirm the IRI therapeutic activity of the PB-MSC cells of the present invention. At this time, the dotted line denotes the necrotic site.

Referring to FIGS. 6 to 7, it can be seen that the necrotic area of the PB-MSC-treated liver tissue is only 1.7±1.4% and is lower than that of the PBS group, the control group, by almost 33 times, and apoptosis is also the lowest in the case of being treated with PB-MSCs. Through this, it has been confirmed that the PB-MSCs of the present invention exhibit improved treatment efficiency of a tissue damage by ischemia-reperfusion compared to that of the MSCs of Comparative Example.

### <Experimental Example 7-3> Experiment to Confirm In Vivo Antioxidant Activity

An experiment was conducted to confirm the in vivo antioxidant activity of the PB-MSCs of the present invention. Excessive ROS production during I/R injury causes severe oxidative injury to lipids, proteins, and DNA. Therefore, lipid peroxidation may be used as a biomarker to evaluate oxidative damage due to I/R injury. In Experimental Example 7-3 of the present invention, the oxidative stress reducing effect and in vivo antioxidant activity of PB-MSCs were confirmed by measuring the level of lipid peroxidation in damaged liver extracts by analysis of thiobarbituric acid reactive substances (TBARS), an end product of lipid peroxidation.

To this end, frozen liver tissue was first suspended in cold RIPA buffer and homogenized. The homogenate was centrifuged at 3000 rpm for 10 minutes at 4° C. and the supernatant was collected for analysis. With 100 μL of 10% (w/v) trichloroacetic acid and 800 μL of thiobarbituric acid (TBA) reagent, 100 μL of the supernatant was mixed and incubated at 95° C. for 60 minutes in a water bath. The amount of TBARS was determined by fluorescence measurement at excitation and emission wavelengths of 530 and 555 nm, respectively. A standard curve was created using pure malondialdehyde (MDA) at various concentrations for quantification. The levels of TBARS in all tissue samples were normalized by measuring total protein using the BCA Protein Assay Kit (Thermo Fischer, Waltham, Mass., USA) and BSA as a standard.

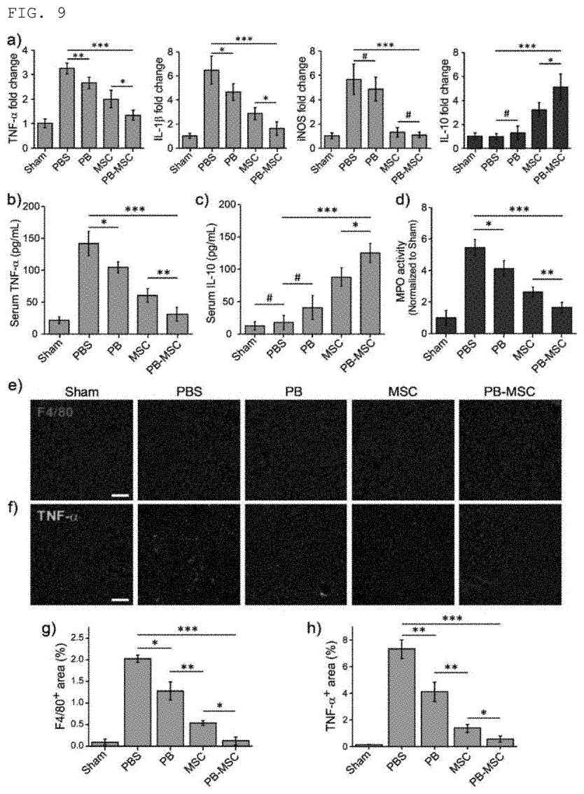

FIG. 8 is images and graphs for confirming the in vivo antioxidant activity of the PB-MSC cells of the present invention.

(a) of FIG. 8 is images illustrating human nuclear antigen (HNA) in liver sections through immunohistochemical staining.

Referring to (a) of FIG. 8, since human MSCs were administered to mice, immunohistochemical staining of human nuclear antigen (HNA) can be used to confirm the presence of transplanted MSC cells in the liver tissue. At this time, it can also be confirmed that there is no false-positive signal through the fact that the Sham, PBS, and PB groups in which MSC cells are not transplanted do not exhibit positive staining for HNA.

(b) of FIG. 8 is a graph illustrating the average number of (HNA⁺) cells per section quantified by image analysis.

Referring to (b) of FIG. 8, it can be seen that the average number of HNA⁺ cells per field is higher in the PB-MSC group compared to the MSC group of Comparative Example. Through this, it has been confirmed that the viability of transplanted PB-MSCs is improved compared to that of MSCs.

(c) of FIG. 8 is a graph illustrating the analysis results of lipid peroxidation in a liver tissue homogenate through TBARS analysis.

Referring to (c) of FIG. 8, the the lipid peroxidation level in the liver extract was measured through TBARS analysis to measure ROS-mediated tissue damage, as a result, oxidative injury of the tissue has been confirmed through the fact that the TBARS value is increased by about 4 times in the PBS group compared to the sham group. The group using PB nanoparticles themselves also decreases the level of lipid peroxidation from the PBS group, and this indicates that PB nanoparticles alone can also inhibit ROS-induced tissue injury to some extent by their ROS scavenging ability. However, the PB-MSC-treated group most powerfully inhibits lipid peroxidation compared to all other groups. Through this, it has been confirmed that the impregnation of MSCs with PB nanoparticles improves the in vivo antioxidant activity of the cells.

### <Experimental Example 7-4> Experiment to Confirm In Vivo Anti-Inflammatory Activity Through Gene Expression Analysis

An experiment was conducted to confirm the in vivo anti-inflammatory activity of the PB-MSCs of the present invention. In addition to the production of a large amount of ROS, another feature of I/R injury is a strong inflammatory response. In the early stage of reperfusion, a large number of neutrophils infiltrate the liver tissue, and Kupffer cells activated together with the infiltrated neutrophils create an inflammatory environment in the tissue. Therefore, the immunomodulatory and anti-inflammatory activity of MSCs at this time is important for the treatment of I/R injury. In order to analyze such anti-inflammatory action of MSCs, the liver expression of TNF-α, IL-1b, iNOS, and IL-10 was compared with one another in Experimental Example 7-4 of the present invention.

To this end, RNA was first purified from a mouse liver tissue using TRIzol reagent, and then RT-qPCR was performed. β-actin was used for normalization of target genes (TNF-α, IL-1βiNOS, IL-10), and the fold change was calculated by the ΔΔCt method in comparison with the Sham group. The primer sequences of the target genes are presented in Table 2 below.

TABLE 2

| Gene | Primers |
|---|---|
| TNF-α | Forward: 5′-CCCTCACACTCAGATCATCTTCT-3′ |
| (mouse) | Reverse: 5′-GCTACGACGTGGGCTACAG-3′ |
| IL-1β | Forward: 5′-CTCCATGAGCTTTGTACAAGG-3′ |
| (mouse) | Reverse: 5′-TGCTGATGTACCAGTTGGGG-3′ |
| iNOS | Forward: 5′-CAGCTGGGCTGTACAAACCTT-3′ |
| (mouse) | Reverse: 5′-CATTGGAAGTGAAGCGTTTCG-3′ |
| IL-10 | Forward: 5′-CCAAGCCTTATCGGAAATGA-3′ |
| (mouse) | Reverse: 5′-TTTTCACAGGGGAGAAATCG-3′ |
| β-actin | Forward: 5′-CTTTGCAGCTCCTTCGTTGC-3′ |
| (mouse) | Reverse: 5′-ACGATGGAGGGGAATACAGC-3′ |

FIG. 9 is graphs and images for confirming the in vivo anti-inflammatory activity of the PB-MSCs of the present invention.

(a) of FIG. 9 is graphs illustrating the expression of pro-inflammatory genes (TNF-α, IL-1βiNOS) and anti-inflammatory genes (IL-10) in the liver.

Referring to (a) of FIG. 9, it can be seen that at the gene level, the expression of pro-inflammatory cytokine genes (TNF-α, IL-lb and iNOS) is significantly higher in the PBS-treated mice compared to the Sham group. The expression of TNF-α and IL-lb is slightly decreased in the PB-treated group, but the expression of iNOS is not affected. In comparison, the treatment with MSCs and PB-MSCs exhibits a stronger inhibitory effect on the expression of TNF-α, IL-lb and iNOS. However, the PB-MSC-treated group exhibits significantly (p<0.05) higher inhibition of TNF-α and IL-lb expression compared to the MSC-treated group. It can be seen that the liver expression of IL-10, an anti-inflammatory cytokine that helps suppress uncontrolled inflammation and promotes hepatocyte proliferation, is greatly increased by 3.2 times and 5.1 times in the MSC group and the PB-MSC group, respectively.

(b) and (c) of FIG. 9 are graphs illustrating the quantified serum concentrations of TNF-α and IL-10, respectively.

Referring to (b) and (c) of FIG. 9, similar to gene expression analysis, it can be seen that the treatment with PB-MSCs has the highest effect in lowering the level of pro-inflammatory TNF-α and increasing the level of anti-inflammatory IL-10.

Myeloperoxidase (MPO) is an enzyme mainly found in neutrophils. Therefore, MPO activity in the liver may be used as a biomarker for neutrophil infiltration in the liver during reperfusion. In Experimental Example 7-4 of the present invention, MPO activity in the liver tissue homogenate was analyzed to confirm the degree of neutrophil infiltration.

To this end, the obtained liver tissue (approx. 50 mg) was first homogenized in 500 μL potassium phosphate buffer (50 mM, pH 6.0) containing 0.5% (w/v) hexadecyltrimethylammonium bromide in an ice bath. The homogenate was centrifuged at 12000 rpm for 15 minutes at 4° C. and the supernatant was collected for analysis. MPO activity of the supernatant was measured by colorimetric analysis at 450 nm using o-dianisidine dihydrochloride as a substrate. The absorbance values were normalized by measuring total protein in the samples and indicated as a fold change with respect to the Sham group.

(d) of FIG. 9 is graphs illustrating MPO activity in the liver measured after the treatment with various groups.

Referring to (d) of FIG. 9, it can be seen that MPO activity is increased by about 5.5 times in the PBS group compared to the Sham group and this indicates significant neutrophil infiltration. Compared to other groups, MPO activity is significantly lower in the PB-MSC group, and this means that MSCs impregnated with PB nanoparticles are most effective in decreasing neutrophil infiltration.

(e) and (f) of FIG. 9 illustrate immunohistochemical staining images of (e) F4/80 (red) using DAPI (blue) and (f) TNF-α (green) using DAPI (blue) in liver sections, respectively. (g) and (h) of FIG. 9 are graphs illustrating the expression level quantified from each of the images.

Referring to (e) to (h) of FIG. 9, it can be seen that minimal macrophage activation leads to low staining of F4/80 in the Sham group, and the significant activation of macrophages in the damaged tissue is confirmed through a far stronger fluorescence signal in the PBS group. As expected, the expression of F4/80 is most greatly decreased in the PB-MSC group. It can be seen that TNF-α, a pro-inflammatory cytokine secreted by activated macrophages, also exhibits the lowest expression in the PB-MSC group. Through the expression measurement of inflammatory genes as described above, it has been confirmed that the PB-MSCs of the present invention exhibit a higher level of anti-inflammatory activity than MSCs.

According to an embodiment of the present invention, there is an effect capable of providing a stem cell therapeutic agent having improved therapeutic performance and improved oxidative stress resistance, engraftment rate and viability at a damage site through the introduction of Prussian blue nanoparticles having ROS scavenging ability and anti-inflammatory properties.

According to an embodiment of the present invention, there is an effect capable of providing a method for preparing a stem cell therapeutic agent for anti-inflammatory or damaged tissue regeneration, which can be simply prepared by only incubating stem cells and Prussian blue nanoparticles together without a special preparation method or cumbersome process.

The effects of the present invention are not limited to the effects, but it should be understood to include all effects that can be inferred from the configuration of the invention described in the detailed description or claims of the present invention.

The foregoing description of the present invention is for purposes of illustration, and those of ordinary skill in the art to which the present invention pertains will understand that the invention can be easily modified into other specific forms without changing the technical spirit or essential features of the present invention. Therefore, it should be understood that the embodiments described above are illustrative in all respects and not restrictive. For example, each component described as a single type may be implemented in a distributed form, and likewise components described as a distributed type may be implemented in a combined form.

The scope of the present invention is indicated by the following claims, and all changes or modifications derived from the meaning and scope of the claims and their equivalents should be construed as being included in the scope of the present invention.

What is claimed is:

1. A cell therapeutic agent for anti-inflammatory or damaged tissue regeneration comprising:
Prussian blue nanoparticles; and
cells.

2. The cell therapeutic agent for anti-inflammatory or damaged tissue regeneration according to claim 1, wherein the Prussian blue nanoparticles exist by being impregnated into an inside of the cells.

3. The cell therapeutic agent for anti-inflammatory or damaged tissue regeneration according to claim 1, wherein
the Prussian blue nanoparticles have ROS scavenging properties, and
the cells have oxidative stress resistance improved by the Prussian blue nanoparticles.

4. The cell therapeutic agent for anti-inflammatory or damaged tissue regeneration according to claim 1, wherein the cells are stem cells.

5. The cell therapeutic agent for anti-inflammatory or damaged tissue regeneration according to claim 1, wherein the Prussian blue nanoparticles have an average particle diameter of 10 nm to 200 nm.

6. The cell therapeutic agent for anti-inflammatory or damaged tissue regeneration according to claim 1, wherein the cells are incubated at a concentration of 25 μg/mL to 500 μg/mL of the Prussian blue nanoparticles.

7. The cell therapeutic agent for anti-inflammatory or damaged tissue regeneration according to claim 1, wherein the cells are incubated together with the Prussian blue nanoparticles for 3 hours to 48 hours.

8. The cell therapeutic agent for anti-inflammatory or damaged tissue regeneration according to claim 1, which is an injection formulation.

9. A method for preparing a cell therapeutic agent for anti-inflammatory or damaged tissue regeneration, the method comprising:
preparing Prussian Blue nanoparticles;
incubating the Prussian blue nanoparticles and cells together so that the Prussian blue nanoparticles are impregnated into an inside of the cells; and
obtaining cells containing Prussian blue nanoparticles from the step.

10. The method for preparing a cell therapeutic agent for anti-inflammatory or damaged tissue regeneration according to claim 9, wherein the cells are incubated at a concentration of 25 μg/mL to 500 μg/mL of the Prussian blue nanoparticles in the step of incubating the Prussian blue nanoparticles and the cells together.

11. The method for preparing a cell therapeutic agent for anti-inflammatory or damaged tissue regeneration according to claim 9, wherein the cells are incubated together with the Prussian Blue nanoparticles for 3 hours to 48 hours in the step of incubating the Prussian blue nanoparticles and the cells together.
